(19) United States
(12) Patent Application Publication
(10) Pub. No.: US 2010/0087698 A1
(43) Pub. Date: Apr. 8, 2010

(54) REPETITIVE TRANSCRANIAL MAGNETIC STIMULATION FOR MOVEMENT DISORDERS

(75) Inventor: Ross G. Hoffman, Grand Junction, CO (US)

Correspondence Address: SWANSON & BRATSCHUN, L.L.C., 8210 SOUTHPARK TERRACE, LITTLETON, CO, 80120, US

(73) Assignee: NEUROQUEST THERAPEUTICS, Grand Junction, CO (US)

(21) Appl. No.: 12/440,708

(22) PCT Filed: Sep. 11, 2007
(86) PCT No.: PCT/US07/78094

§ 371 Date: Nov. 18, 2009

Related U.S. Application Data
(60) Provisional application No. 60/825,222, filed on Sep. 11, 2006.

(51) Int. Cl. A61N 2/02

(52) U.S. Cl. 600/13

(57) ABSTRACT

Methods are disclosed for using transcranial magnetic stimulation to treat a particular muscle grouping involved in a movement disorder comprising treating a patient with a treatment course comprising a plurality of treatment regimens, each regimen comprising applying repetitive transcranial magnetic stimulation (rTMS) over a position on the patient's scalp corresponding to a cortical region controlling the muscle grouping.

### TECHNICAL FIELD

A method of using repetitive transcranial magnetic stimulation (rTMS) to treat a muscle group involved in a movement disorder.

### BACKGROUND

Electricity and magnetism are interdependent (Maxwell's equations). Passing current through a coil of wire generates a magnetic field perpendicular to the current flow in the coil. If a conducting medium, such as a brain, is adjacent to the magnetic field, electrical current will be induced in the conducting medium. The low of the induced current will be parallel, but opposite in direction, to the current in the coil. Thus, transcranial magnetic stimulation (hereinafter TMS) has been referred to as electrode-less electrical stimulation to emphasize that the magnetic field acts as the medium between electricity in the coil and induced electrical currents in the brain.

TMS involves placing a electromagnetic coil near or on the scalp. Subjects are awake and alert. High intensity current is rapidly turned on and off in the coil through the discharge of capacitors. This produces a time varying magnetic filed that lasts for about 100-200 microseconds. The magnetic field typically has a strength of between 0.5 and 2 Tesla. The proximity of the brain to the time varying magnetic filed results in current flow in neural tissue.

TMS of sufficient intensity over the premotor cortex, primary motor cortex, or supplementary motor area will cause involuntary movement. The magnetic field intensity needed to produce motor movement varies considerably across individuals and is known as the motor threshold. Placing the coil over different areas of the above-identified cortical areas causes contralateral movement in different distal muscles. TMS can be used to map the representation of body parts in the motor cortex on an individual basis. It is known that repeated rhythmic TMS (known as rTMS) can cause interference with or augmentation of information processing and neuronal behavior. If the stimulation occurs at or less than about once per second (1 Hz), inhibition of the motor cortex is produced.

TMS evoked motor responses result from the direct excitation of corticospinal neurons at or close to the axon hillock. It is thought that the TMS magnetic field induces an electrical current in superficial cortex. The TMS magnetic field declines exponentially with distance from the coil. This limits the area of depolarization to a depth of about 2 cm below the brain's surface. The peak effect of TMS can be localized to within less than a millimeter in terms of functional location. Nerve fibers that are parallel to the TMS coil (perpendicular to the magnetic field) are more likely to depolarize than those perpendicular to the coil. Conventional TMS coils are either round, or in the shape of a figure eight. The figure eight designs are more focal than the round coils.

Movement disorder is a problem affecting up to forty million individuals in the United States. There is a broad range of movement disorders, including ones of idiopathic origin or secondary to other injury or disease process, such as stroke, traumatic injury to the brain, or diseases such as Parkinson's and the like. Dystonia is one such movement disorder. Dystonia can be most generally divided into generalized dystonia (affecting more than one muscle group) or focal dystonia (affecting one muscle group). Generalized dystonia typically manifests most strongly in one particular muscle group. Focal dystonias range from writers' cramp to the golfer's ailment YIPS, to torticollis (contraction of the muscles of the neck causing abnormal posture), spasmodic dysphonia causing difficulties with speech, and dystonia of the foot and lower extremity. Often generalized dystonia will manifest first in the most distal muscle groups leading to dystonia of the foot and lower extremity. DYT1 dystonia is the most common form of hereditary dystonia, a dominantly inherited disorder with variable penetrance. The causes of dystonia are not well understood, although a number of groups have postulated that reduced effectiveness of intracortical inhibitory circuits may play a role. Some groups have theorized that particularly in genetically susceptible individuals, overuse or repetitive trauma may modify the characteristics of sensory inputs from a specific body area leading to an abnormal sensorimotor integration or even to plastic cortical reorganization. An increased synaptic connectivity might be responsible for the increased input/output relationship in dystonics. In particular, sensory inputs might be abnormally processed in the brain of dystonic patients with a defective activation of local cortical inhibitory systems. This would in turn increase the excitability of the motor cortical areas leading to an inappropriate output and more widespread muscle activation.

Primary movement disorders are almost always progressive, leading to loss of function and personal independence. Secondary cases such as those due to stroke or cerebral palsy, though sometimes not progressive, are often disabling. In primary cases, cessation or slowing of progression would be considered a significant therapeutic advance, one which may allow patients to diminish their dosages of medications.

Current therapies for movement disorders including focal dystonia include injections of anesthetic agents (such as, for example, lidocaine or ethanol) or nerve blocking agents (such as, for example, botulinum toxin including BOTOX, Allergan Pharmaceuticals) into afferent nerve roots in the affected muscle groups. Nerve blocking agents have the side effect of causing loss of function in a patient, an undesired side effect. Anesthetic agents cause only a transient improvement in dystonic symptoms, and administration is inconvenient and uncomfortable for the patient.

Recently it has been suggested that rTMS at low frequencies of the premotor cortex and supplementary motor area may reduce cortical excitability in patients with dystonia causing corresponding changes in abnormal spinal motor output. (Huang et al. Movement Disorders Vol. 19, 54-59 (2004)). These authors note that rTMS causes only “short-lived reductions in cortical excitability”. rTMS was performed for one session of 1200 pulses at 1 Hz. The authors note that previous studies to determine possible therapeutic effects of rTMS in dystonia have been disappointing with any benefits to have been slight and extremely transient, and notes that in its current form, rTMS is not likely to have therapeutic implications for those with dystonia. In another study, looking at rTMS effects on regional cerebral blood flow (rCBF) as a marker of synaptic activity, the authors found that synaptic activity can be reduced by 1 Hz rTMS for up to one hour after the end of stimulation. Siebner et al. Brain Vol. 126, 2710-2725 (2003). These authors note that reliable therapy would need to produce longer lasting effects than the ones seen in the study. Despite interest from researchers and clinicians, and initial optimism, the generally accepted view is that rTMS will not provide the longer lasting effects which are required for practical therapeutic application. Effecting only transient symptom reduction, rTMS has not been shown to alter the clinical course of progressive movement disorders such as dystonia.

Accordingly, a need in the art remains for more effective treatments and therapeutically relevant rTMS procedures for relieving the symptoms of movement disorders, particularly dystonic movement disorders, which are non invasive in nature and cause non-transient effects and result in lasting clinical efficacy.

### SUMMARY OF THE INVENTION

A method of using transcranial magnetic stimulation (rTMS) to treat a muscle group involved in a movement disorder. The method includes treating a patient with a treatment course having more than one treatment regimen where each treatment regimen comprises applying rTMS over a position on the patient's scalp corresponding to a cortical region controlling the muscle group. The movement disorders which are treatable with the disclosed methods include but are not limited to: local dystonia, generalized dystonia, post-stroke movement disorder, Parkinsonian tremor and essential tremor. If the movement disorder treated with the method is generalized or focal dystonia, the focus may be on the hand, neck, vocal chords or other parts of the patient's body. The movement disorder may be writer's cramp, Yips, torticollis, spasmodic dysphonia or other movement disorders. The treatment course may include at least two or more treatment regimens rendered over a select period of time. The select period of time may be one or multiple seven-day periods.

The method may include adjusting the frequency of the rTMS stimulation to provide an inhibitory effect to motor activation by the cortex. The frequency of the rTMS may be between 0.2 Hz and 1 Hz. The duration of the rTMS treatment regimen may be adjusted to accomplish specific therapeutic goals. The duration of a treatment may range from about 10 minutes to about 120 minutes.

### BRIEF DESCRIPTION OF THE DRAWING

FIG. 1 is a schematic diagram of a 10-20 electrode position map consistent with the present invention.

### DETAILED DESCRIPTION

The present disclosure is based on the surprising discovery that rTMS applied in a treatment course comprising a plurality of treatment regimens over the course of time can be therapeutically relevant and provide non-transient, longer lasting and/or more sustained relief of symptoms of a movement disorder such as dystonia. Conventionally, it would be expected that since single-application rTMS yields only transient (one hour at most) symptom reduction in dystonic symptoms, that multiple applications of rTMS to the relevant cortical areas for a particular movement disorder would similarly yield a series of transient effects with no generalized improvement in the course of or symptoms of the movement disorder or slowing of the clinical progression of any underlying disease, such as, for example, generalized dystonia. However, the present inventor has found that surprisingly, a series of treatment regimens comprising rTMS applications over time leads to an overall improvement in symptoms of the movement disorder and/or reduction in the time course of the underlying disease, such as dystonia. Accordingly, the present invention comprises a method for using rTMS for treatment of a movement disorder comprising treating a patient with a treatment course comprising a plurality of treatment regimens comprising application of rTMS to a particular cortical region corresponding to the affected muscle grouping.

One embodiment includes a method for using transcranial magnetic stimulation to treat a particular muscle grouping involved in a movement disorder. The method includes treating a patient with a treatment course which includes a plurality of (i.e., more than one) treatment regimens. Each treatment regimen includes applying repetitive transcranial magnetic stimulation (rTMS) over a position on the patient's scalp corresponding to a cortical region controlling the muscle grouping.

A number of movement disorders may be treated with the methods disclosed herein. A partial list of movement disorders to treat include ataxia, bradykinesia, choreoathetosis, corticobasal degeneration, dyskinesias (paroxysmal), dystonia, essential tremor, hereditary spastic paraplegia, Huntington's disease, multiple system atrophy, myoclonus, Parkinson's disease, progressive supranuclear palsy, restless legs syndrome, Rett syndrome, spasticity, sydenham's chorea, tardive dyskinesia, tics, Tourette's syndrome, tremor and Wilson's disease. Particularly preferred movement disorders to treat include tremor, dystonia, and parkinsonism. In one embodiment, the movement disorder is a focal dystonia. Examples of focal dystonias to treat include a hand or arm dystonia, such as writer's cramp or YIPS (a golfer's disorder); a vocal cord dystonia such as spasmodic dysphonia; a neck or cervical dystonia such as torticollis; or a foot or lower extremity dystonia. In another embodiment, the movement disorder is a generalized dystonia. Generalized dystonia generally manifests first in a distal muscle group such as the foot or lower extremity. Generalized dystonia can be treated by methods of the current invention by choosing a particular muscle grouping or groupings that are most affected by dystonia, which can include any of the muscle groupings involved in the focal dystonias listed above or others not specifically listed. Preferably, a generalized dystonia or focal dystonia to treat is characterized as a “mild” or “moderate” generalized dystonia or focal dystonia by established clinical criteria.

It has also been determined that a number of other conditions may be treated with the methods disclosed herein. These conditions include but are not limited to autism and stroke recovery from stroke injuries resulting in motor deficits, Parkinson's disease, tinnitus, Bell's palsy, Tourette's syndrome, epilepsy, traumatic brain injury, persistent vegetative state, cerebral palsy, depression, anxiety disorders and mood disorders but not limited to, cerebral palsy, attention deficit disorder (ADD), autism, Asperger's, Learning disabilities (dyslexia, dysgraphia, dyspraxia, clumsiness), nonverbal learning disabilities (dyscalculia, visuospacial dysfunction, socioemotional disabilities, ADHD); Demyelinating diseases (multiple sclerosis); delirium and dementia; affective disorders; substance abuse-related disorders; sexual dysfunctions; eating disorders; anxiety and OCD; impulse control disorders; personality disorders; traumatic brain injury (TBI) including related pain syndromes such as fibromyalgia, related cognitive deficits and/or behavioral aberrations (for example depression, emotional distress, impaired short term memory, visual and auditory memory), reduced attention and concentration and reduced information processing capacity and migraine or complex cluster headaches.

In certain embodiments, rTMS may be used in combination with other forms of stimulation such as TDCS (transcranial direct current stimulation), thermal stimulation or ultrasound administration. TDCS is a form of very low energy brain stimulation performed by creating a weak electrical field against the scalp. TDCS and other combination approaches may be used to enhance the therapeutic impact of the rTMS. Methods of the present invention result in improvements in symptom reduction and/or clinical course of the underlying diseases compared with current therapies.

A treatment course according to the disclosed methods will preferably comprise a number of treatment regimens over a several week period. More specifically, a treatment course will, in one embodiment, comprise at least two treatment regimens over the course of one week (7 day period), comprise at least three treatment regimens over the course of a week, comprise at least four treatment regimens over the course of a week, or comprise at least five treatment regimens over the course of a week. More treatments per week, including at least six treatments, at least seven treatments, at least eight treatments, at least nine treatments, at least ten treatments, at least twelve treatments, at least fourteen treatments, and at least sixteen treatments, are also included in the present invention. The higher number of treatments per week may result from more than one treatment per day. Accordingly, treatment regimens of the present invention include more than one treatment regimen per day. In one embodiment, two, three, four or even more treatments may be administered per day.

In one embodiment, timing of the regimens throughout the week can vary, and also can be administered over any sequence of days throughout the week. Where the 7 day period is defined as Sunday through Saturday, in one embodiment, where the treatment course is two days per week, the treatments will occur preferably on a Monday and Thursday. Where the treatment course is three days per week, the treatments will occur preferably on a Monday, Wednesday, and Friday. Where the treatment course is five days a week, the treatments will occur preferably on a Monday, Tuesday, Wednesday, Thursday, and Friday of the week.

In one embodiment, numerous treatment regimens are administered. The minimum number of total treatment regimens is at least two, but more are preferred. Preferably at least about four, at least about six, at least about eight, at least about ten, at least about twelve, at least about fourteen, more preferably at least about sixteen, at least about eighteen, at least about twenty, at least about twenty two, at least about twenty four, at least about twenty six, at least about twenty eight, at least about thirty, at least about thirty two, at least about thirty four, at least about thirty six, at least about thirty eight, even more preferably at least about forty, at least about forty two, at least about forty four, at least about forty eight, at least about fifty treatment or more regimens are performed in a treatment course. In another embodiment, at least about fifty four, at least about fifty eight, at least about sixty, at least about sixty five, at least about seventy, at least about seventy five, at least about eighty, at least about eighty five, at least about ninety, and at least about ninety five treatment regimens are performed in a treatment course. Even more treatment regimens may be included in the treatment courses of the present invention, but limitations on funding, reimbursement and clinical effectiveness for a higher number of visits may exert an upper limit on the number of treatment regimens. In one embodiment, the treatment course comprises the minimum number of weeks that result in a desired improvement or maximum improvement in symptoms and/or course of progress of the movement disorder.

The treatment course is, in one embodiment, organized into treatment regimens for a plurality of 7 day periods (weeks). In one embodiment, a treatment course include at least about two weeks, at least about three weeks, at least about four weeks, at least about six weeks, at least about eight weeks, at least about ten weeks, at least about twelve weeks, at least about fourteen weeks, at least about eighteen weeks, at least about twenty weeks, at least about twenty two weeks, at least about twenty four weeks, at least about twenty six weeks, at least about twenty eight weeks, at least about thirty weeks, at least about thirty two weeks, at least about thirty four weeks, and at least about thirty six weeks. In one embodiment the treatment course is about twelve weeks or more and about twenty four weeks or more. Treatment courses may be repeated either identical to the earlier treatment course or a new treatment course may be designed, depending on clinician assessment.

In another embodiment, a treatment course includes a first portion which will have a higher frequency of treatment regimens followed by a second portion which will have a lesser frequency of treatment regimens. Any variations of treatment courses which include a first portion and a second portion which result in the desired and/or maximum improvement in a cost effective manner are preferred. Particularly preferred is a treatment course which includes a first portion including at least about three treatment regimens per week for a period of weeks ranging from between about four weeks and about twelve weeks, followed by a second portion including two treatment regimens per week for a period of weeks ranging from between about four weeks and about twelve weeks. Another particularly preferred treatment course includes a treatment regimen which includes a first portion including at least about five treatments per week for a period of weeks ranging between about four weeks and about twelve weeks, followed by a second portion including two treatment regimens per week for a period of weeks ranging from between about four weeks and about twelve weeks. Generally speaking, the methods of the present invention can be re-initiated upon any decline in improvement noted or worsening of symptoms.

An rTMS system suitable for the present invention includes TMS units made by a number of manufacturers and include the MAGSTIM rapid stimulator connected to four booster modules (Magstim Company Ltd, Whitland, U.K.); the MAGSTIM 200 and Magstim QuadroPulse Model 500 made by the same manufacturer, and the MAGPRO stimulator (Medtronic-Neuromuscular, Skovlunde, Denmark). The principles underlying TMS are well known. Briefly, a time varying current in a primary circuit (the coil) will induce an electric field and thereby a current flow in the brain. The interaction is mediated by the magnetic field generated by the changing current in the coil. At a microscopic level, the electric field affects the transmembrane potential, which may lead to local membrane depolarization and subsequent neural activation and/or inhibition. rTMS is known to either activate or suppress motor or sensory function, depending on the brain location for that motor or sensory function and parameters of rTMS delivery.

Macroscopic responses to rTMS can be detected with functional imaging tools such as electroencephalography, positron emission tomography, functional magnetic resonance imaging, motor electron potentials, or clinical changes. A more detailed discussion of an exemplary rTMS unit is contained within, for example, U.S. Patent Application Publication No. 20050154426; and U.S. Patent Application Publication No. 20050256539; all of which are incorporated by reference herein in their entireties. Generally, the circuit used consists of a discharge capacitor connected with the coil in series by a thyristor. The capacitor is initially charged to 2-3 kV then discharged through the coil as the gating of the thyristor converts to the conducting state. The current that is generated lasts about 300 microseconds (also known as the pulse width) with a peak value of 10 kA, for example, which creates a magnetic field strength of about 1 tesla. The shape of the electric field that is generated is dependent on factors such as the shape of the induction coil, the location and orientation of the coil with respect to the scalp, and the electrical conductivity of the tissue. The simplest shaped coil is a circular one, with round coils being relatively powerful, but they have a larger focal point than the figure of eight shaped coils or butterfly shaped coils which elicit a maximal current at the intersection of the two round or oval components. Preferably the coil is a figure of eight shaped coil.

It has been found that different frequencies of magnetic stimulation will have different effects. In one embodiment, the magnetic field's properties are adjusted such that upon treatment, inhibitory effects are seen in the patient's motor cortex. Typically, such effects are seen with rTMS frequency between 0.2 and 1 Hz (cycles per second). Preferably, the frequency is between about 0.2 and 0.5 Hz. The electrical current used for generating the magnetic field may be any that is known in the art, and preferably is either monophasic, biphasic, or polyphasic. Monophasic or biphasic is preferred. The intensity of the electrical field to use to generate the magnetic force can vary. As a rough guide, intensities of about 1 tesla (T) with an upper limit of about 2 T are typically generated.

Preferably, the intensity of electrical field used to generate the magnetic field (and generate the eddy current in the brain) is about 90% of the motor threshold for the particular muscle grouping to be treated. Motor threshold is a measure that varies considerably between subjects due to factors such as skull thickness, head shape, cortical excitability, medication and acute brain state. Gauging the motor threshold of the subject enables comparable strengths of stimuli to be employed between patients leading to more predictable therapeutic outcomes. Methods by which to determine motor threshold are known in the art, and include determining the minimum stimulation intensity over a motor hot spot that can elicit an motor evoked potential (MEP) of no less than 50 microV in 5 of 10 trials. MEP may be recorded with subdermal needle electrodes in a tendon-belly arrangement via electromyogram (EMG) measurement. EMG measurements may be taken by applying conductive elements or electrodes to the skin surface, or invasively within the muscle. Surface EMG is the more common method of measurement, since it is non-invasive. The EMG signals are amplified and filtered to graphically record and quantify the degree of muscle activity. The microvoltage of EMG measurement is directly proportional to the mechanical muscle contraction. EMG measurements are useful to both help confirm coil positioning and assess cerebral cortical excitability. In practice, typically the electric field strength will be of the order of about 100 mV/mm to elicit sufficient motor cortex activation leading to muscle twitches. With the conductivity of the brain being about 0.4 S/m, the corresponding cortical current density will typically be approximately 40 A/mm².

Current direction and coil direction are other variables that one of skill in the art can vary depending on the subject. The number of pulses to deliver may also be determined by one of skill in the art. Preferably, the number of pulses to deliver comprises the number of pulses capable of being delivered in a time period of between about ten minutes and about 120 minutes, between about twenty minutes and about ninety minutes, or most preferably between about thirty minutes and about sixty minutes. The number of pulses delivered in that time period can be determined with a simple calculation. For example, the number of pulses delivered at a frequency of 0.5 Hz will preferably comprise between about five pulses and about sixty pulses, between about ten pulses and about forty five pulses, or most preferably between about fifteen pulses and about twenty pulses. Frequency may change during a particular treatment regimen and/or during a treatment course.

Optimal positioning of the coil over the scalp may readily be determined by one of skill in the art, and will generally comprise positioning to correspond with the cortical region associated with the particular muscle grouping for which treatment is desired in accordance with the Ten-Twenty (10-20) System, which is a system of locating positions (sites) on the scalp, for standard EEG recording. See Jasper H H. The Ten Twenty Electrode System of the International Federation. Clinical Neurophysiology 1958;10:371-375. They are based on 10% and 20% of the distance across the head, hence the name. Standard charts and descriptions of the locations are freely available. In order to determine the proper scalp positioning to maximal stimulation of a particular brain coordinate, a number of algorithms known in the art may be used, for example, the T2T-Converter (Talairach-to-Ten-Twenty-Converter) which calculates the optimal stimulation positions for TMS studies by projecting brain coordinates to scalp coordinates. Given a Talairach coordinate specifying a brain point, the converter searches for the closest scalp position perpendicular to this point. As reference model for the scalp, the MRI template ICBM152 provided by the Montreal Neurological Institute is used. This is identical to the T1 template released by SPM99, and therefore, brain points can also be specified as MNI coordinates (the standard coordinate system of SPM). A non-linear transformation is used to convert between Talairach and MNI coordinates. For the resulting scalp positions, a 2D coordinate system (10-20 coordinates) based on the International Ten-Twenty System for EEG electrode placement is used.

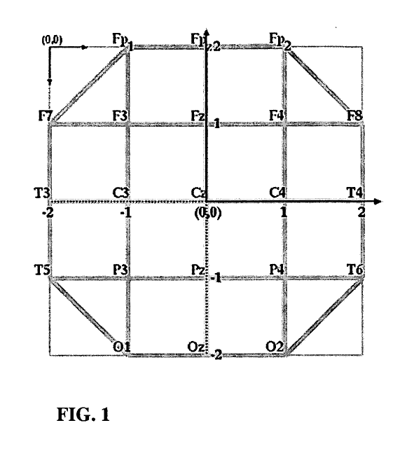

As shown in FIG. 1, the portion of the scalp covered by the Ten-Twenty System is mapped to [−2,2]² so that Cz is mapped to to the origin of the coordinate system and each other Ten-Twenty electrode position is mapped to an integral coordinate. Any other point in this scheme can be mapped back to the scalp by interpreting its 2D coordinate relative to the locations of the surrounding Ten-Twenty electrodes. For instance, (−0.5, −1.5) can be located on the scalp by drawing a line from T3 to T5 as well as a line from C3 to P3. The center (referring to x=−1.5) of a third line going from the center (referring to x=−0.5) of the first line to the center (again x=−0.5) of the second line yields the searched scalp position (Cp5).

rTMS may be applied to a variety of cortical regions dependent upon the disorder to be treated. The regions of interest include (but are not limited to) the primary motor cortex, (MC), premotor cortex (PMC) supplementary motor area (SMA) cerebellum and parietal cortex. Scalp positioning of the coils is selected in a manner consistent with currently published data which utilizes anatomically established or image guided (MRI/fMRI/PET) landmarks (with standardized probabilistic coordinates). In one embodiment, after scalp positioning has been calculated, optionally, by using the above-referenced algorithm, the position of the coil of the rTMS device in a treatment regimen for treatment of the brain region corresponding to a particular muscle grouping is determined by locating the motor hot spot in the cortex for the hand area by incremental movements over the scalp to find the “hot spot” where magnetic stimulation provokes the largest motor evoked potential (MEP) of a given muscle group in the hand; and positioning the coil approximately 2.5 cm anterior to the motor hot spot (to correspond with the premotor cortex). The motor cortex areas corresponding to various muscle groupings such as those corresponding to the hand, foot, lower extremities, neck, vocal cords, among others, are closely positioned in the brain. Accordingly, determining the motor “hot spot” for these will often require technicians to carry out studies to find the proper positioning, as discussed above. In addition to the specific methods discussed above, any other method to find the proper positioning for rTMS stimulation of a particular cortical region is included in the present invention. Optionally, one may determine which hemisphere to treat with rTMS depending on the laterality of the movement disorder, e.g., right hand dominant, stimulation is carried out on the left hemisphere of the brain. Stimulation of certain muscle groups may require higher field intensity due to deeper positioning of the control areas in the brain.

In another embodiment, a technique called transcranial direct current stimulation (tDCS) is used in place of rTMS or in addition to rTMS. In this embodiment, the present invention includes a method for using tDCS to treat a particular muscle grouping involved in a movement disorder. The method includes treating a patient with a treatment course which includes a plurality of (i.e., more than one) treatment regimens as is described above for rTMS. Each treatment regimen includes applying tDCS in order to induce an excitability shift towards less excitability of the motor cortex. Preferably, the tDCS is applied in a cathodal modality. tDCS in a particular treatment regimen may be applied as known in the art.

In one embodiment, tDCS comprises the application of very weak electrical currents (1-2 mA (thousandths of an amp)) to modulate the activity of neurons in the brain. Several generations of neurophysiological experiments have shown that neurons respond to static (DC) electrical fields by altering their firing rates. Firing increases when the positive pole or electrode (anode) is located near the cell body or dendrites and decrease when the field is reversed. It is known that electrodes placed on the forehead and back of the head were able to produce noticeable psychological changes that were dependent on the direction of the field. Recently, it was shown by Nitsche et al. (J. Physiol 561.1 pp. 291-303 (2005)) that anodal polarization of the motor cortex increased the motor response of transcranial magnetic stimulation of the same area and reduction of this response was observed with cathodal polarization. It should be noted that the term “rTMS” as used herein includes rTMS as well as tDCS, or a combination of rTMS and tDCS. In other words, the term rTMS includes the techniques known as rTMS, tDCS, and combinations thereof.

DC brain polarization is not “stimulation” in the same sense as transcranial magnetic stimulation or the stimulation of the brain and nerves with conventional electrical techniques. It does not appear to cause nerve cell firing on its own and does not produce discrete effects such as muscle twitches etc., associated with classical stimulation. It is also important to distinguish it from electroconvulsive therapy, which is used to treat mental illnesses such as major depression by passing approximately trains of 1 amp pulses into the brain in order to provoke an epileptic seizure.

Suitable patients to treat with the methods of the present invention include humans; birds such as chickens, ostriches, quail, and turkeys; mammals such as companion animals (including dogs, cats, and rodents) and economic food and/or fur or other product animals, such as horses, cattle, llamas, chinchillas, ferrets, goats, sheep, rodents, minks, rabbits, raccoons, and swine. Humans are preferred.

In one embodiment, the treatment course leads to clinically relevant improvement in a symptom of the movement disorder as measured by one of skill in the art. In one embodiment, clinically relevant improvement is a functional improvement. Functional improvement may be evaluated by any method known in the art, and can be evaluated either subjectively (e.g., patient generated feedback or clinician observation) or objectively (e.g., published dystonia scores, writing samples, the Fahn Dystonia Scale, the Tremor Clinical rating scale, and other rating/evaluation methods or symptoms that are known in the art.) Preferably, improvement is evaluated both subjectively and objectively. Additionally, various markers of improvement are known to those of skill in the art, and include surrogate markers of improvement as well as the clinically relevant improvement(s) as discussed above. Examples of markers of improvement include reduction in synaptic activity as measured by reductions in regional cerebral blood flow, and quantitative and qualitative evaluation of movements by the particular muscle groupings before and after stimulation, and other markers known in the art.

Preferably, after a course of treatment according to the method disclosed herein, the improvement in a symptom or marker of improvement of the movement disorder is a non-transient improvement (for example, a non-transient effect). In one embodiment, a non-transient improvement is an improvement in a symptom or marker lasts for longer than one hour, longer than four hours, longer than eight hours, longer than twelve hours, longer than eighteen hours, longer than twenty four hours, longer than thirty six hours, longer than forty eight hours, longer than sixty hours, longer than seventy two hours, longer than four days, longer than five days, longer than six days, longer than a week, longer than two weeks, longer than three weeks, longer than a month, longer than two months, longer than three months, longer than four months, longer than five months, longer than six months, longer than nine months, and longer than year.

In another embodiment, wherein the movement disorder is progressive or is potentially progressive, the treatment course leads to a slowing in the time course of the progression of the movement disorder. Such slowing of the time course of progression may be evaluated by a clinician using methods known in the art, such as evaluating or monitoring the patient using methods such as clinical assessment and/or electrophysiological testing. In one embodiment, the progression of the movement disorder is slowed by about 1%, 2%, 3%, 4%, 5%, 6%, 7%, 8%, 9%, 10%, 12%, 14%, 16%, 18%, 20%, 22%, 24%, 25%, 30%, 35%, 40%, 45%, 50%, 55%, 60%, 65%, 70%, 75%, 80%, 85%, 90%, and 95%, as compared with no treatment or sham treatment.

The following examples are provided for illustrative purposes only and are not intended to limit the scope of the disclosure.

### EXAMPLES

### Example 1

### Writer's Cramp

Several patients having a mean age of about forty years of age having symptoms pertaining to their ability to write and in other tasks (dystonic writer's cramp) in at least one hand are selected for a study. The average duration of the condition is about eight years. Patients are taking no medication at the time of the study and some of the patients have a history of treatment with local lidocaine for a local afferent block, or with botulinum toxin to inhibit antagonist muscle activity. Studies are carried out at least one week post injection. The inclusion criteria for the study is adult onset with no family history; no response to l-dopa; proximal muscles affected in addition to distal muscles; and ability to hold a pen and write selected sentences. Patients with drug induced dystonia are excluded from the study.

All patients have rTMS at a stimulation site over the premotor cortex or with a sham coil over the premotor cortex. The subjects are seated comfortably in a reclining chair and told to relax. Motor evoked potentials (MEP) are recorded with silver chloride disc electrodes, 1 cm in diameter, placed over the muscle belly and tendon of the right first dorsal interosseous. Electromyography (EMG) signals are amplified, analogue-filtered, by an amplifier, and acquired at a sampling rate of 5 kHz. Monophasic rTMS is applied at 0.2 Hz, and 250 stimuli are delivered to the PMC. A figure of eight stimulation coil is used, and the rTMS unit used is a Magstim 200 stimulator (Magstim, Whitland, UK). The stimulation site for PMC stimulation is determined to be 2 cm anterior and 1 cm medial to the hotspot, estimated from the dorsal PMC established in a PET study as described in Fink et al. 1997 J. of Neurophysiol 77:2164-74. Stimuli are set to 80% of the resting motor threshold. To determine the motor hotspot, the stimulation coil is placed over the area 2 cm anterior to Cz (international 10-20 system) with the handle pointing backward and parallel to the midline. The intensity of stimulation is increased from 30% of the maximum output of the stimulator in 5% steps until an MEP just became visible. The coil is then moved in 0.5 cm steps in four directions (medial, lateral, posterior and anterior) until the maximum MEP was found. The coil is then removed and the position is marked on the subject's scalp as the motor hotspot. The resting motor threshold is defined by decreasing or increasing the stimulus intensity in 1% steps, as the minimum intensity that produces MEPs greater than 50 V.

Patients are treated with a treatment course of a first portion of three treatment regimens per week (Monday-Wednesday-Friday) for a period of twelve weeks, and a second portion including two treatment regimens per week (Tuesday-Thursday) for twelve weeks.

Evaluation of handwriting is assessed using a system designed for measuring voluntary movements of the upper limbs which consists of a pressure sensitive digitizing tablet with a crystal display and personal computer based movement analysis software. A target (1 cm in diameter) appears on the crystal display and subjects are asked to track it with a stylus pen. The axial pressure at the tip (pen pressure component) is also measured. Evaluation is also carried out using the Fahn dystonia scale. Patients are also asked to report the subjective rating of their symptoms after rTMS into five grades: improvement, slight improvement, no change, slight deterioration, and deterioration. Patients are evaluated at the end of each treatment regimen, one hour after the end of each treatment regimen, and ten hours at the end of each treatment regimen. Results are statistically analyzed to determine whether findings are significant. Improvement or slight improvement is reported by the majority of the patients and in 11% of the sham patients and shown in analysis of pen pressure and tracking accuracy where improved tracking and decreased pen pressure are correlated with improvement. Results showed that as the treatment course progresses, the number of patients showing clinical improvement and improvement of pen pressure/tracking accuracy increases. Additionally, responses to rTMS are found to last for longer periods of time post each treatment regimen.

### Example 2

### Spasmodic Dysphonia

Several patients having a mean age of about forty years of age having symptoms pertaining to their ability to their ability to speak are selected for the study (spasmodic dysphonia). Patients describe a slow, progressing form of voice difficulty that consists of voice breaks, some degree of hoarseness, more difficulty speaking in noisy situations, and increased difficulty when attempting to use a loud voice. Acoustic and aerodynamic assessments of the voice are made to determine the severity as well as to objectively identify the presence of voice breaks. Patients are evaluated as to whether they primarily have adductor spasmodic dysphonia (ADSD), characterized by a squeezed, strained-strangled effortful phonation with voice stoppages and voice breaks, or abductor spasmodic dysphonia (ABSD), characterized by an excess flow of air with intermittent lack of vocal fold closure. The average duration of the condition is about eight years. Patients are taking no medication at the time of the study and some of the patients have a history of treatment with local lidocaine for a local afferent block, or with botulinum toxin to inhibit antagonist muscle activity. Studies are carried out at least one week post injection. The inclusion criteria for the study is adult onset with no family history; no response to l-dopa. Patients with drug induced spasmodic dysphonia are excluded from the study.

All patients have rTMS at a stimulation site over the premotor cortex or with a sham coil over the premotor cortex. The subjects are seated comfortably in a reclining chair and told to relax. Positioning is determined by the position over the premotor cortex where the minimal intensity is required to evoke the motor threshold of the vocal fold muscle. Monophasic rTMS is applied at 0.2 Hz, and 250 stimuli are delivered to the PMC. A figure of eight stimulation coil is used, and the rTMS unit used is a Magstim 200 stimulator (Magstim, Whitland, UK). Stimuli are set to 80% of the resting motor threshold. The intensity of stimulation is increased from 30% of the maximum output of the stimulator in 5% steps until an MEP just becomes visible. The coil is then moved in 0.5 cm steps in four directions (medial, lateral, posterior and anterior) until the maximum MEP was found. The coil is then removed and the position is marked on the subject's scalp as the motor hotspot. The resting motor threshold is defined by decreasing or increasing the stimulus intensity in 1% steps, as the minimum intensity that produces MEPs greater than 50 V.

Patients are treated with a treatment course of a first portion of three treatment regimens per week (Monday-Wednesday-Friday) for a period of twelve weeks, and a second portion including two treatment regimens per week (Tuesday-Thursday) for twelve weeks.

Evaluation of spasmodic dysphonia is assessed using a system designed for measuring improvements in voice including acoustic and aerodynamic assessments of the voice. Improvements are seen in the amount of voice breaks, degree of hoarseness, speaking in noisy situations, and ability to use a loud voice. Patients are also asked to report the subjective rating of their symptoms after rTMS into five grades: improvement, slight improvement, no change, slight deterioration, and deterioration. Patients are evaluated at the end of each treatment regimen, one hour after the end of each treatment regimen, and ten hours at the end of each treatment regimen. Results are statistically analyzed to determine whether findings are significant. Improvement or slight improvement is reported by the majority of the patients. Results showed that as the treatment course progresses, the number of patients showing clinical improvement increases. Additionally, responses to rTMS are found to last for longer periods of time post each treatment regimen.

### Example 3

### Torticollis

Several patients having a mean age of about forty years of age having symptoms including spasms causing forward (anterocollis), backwards (retrocollis), and sideways (torticollis) movements of the neck are selected for the study. The movements are sustained and/or jerky. Spasms in the muscles or pinching nerves in the neck result in considerable pain and discomfort in the patients. The average duration of the condition is about eight years. Patients are taking no medication at the time of the study and some of the patients have a history of treatment with local lidocaine for a local afferent block, or with botulinum toxin to inhibit antagonist muscle activity. Studies are carried out at least one week post injection. The inclusion criteria for the study is adult onset with no family history; no response to l-dopa. Patients with drug induced dystonia are excluded from the study.

All patients have rTMS at a stimulation site over the premotor cortex or with a sham coil over the premotor cortex. The subjects are seated comfortably in a reclining chair and told to relax. Motor evoked potentials (MEP) are recorded with silver chloride disc electrodes, 1 cm in diameter, placed over the muscle belly and tendon of the affected neck muscle(s). Electromyography (EMG) signals are amplified, analogue-filtered, by an amplifier, and acquired at a sampling rate of 5 kHz. Monophasic rTMS is applied at 0.2 Hz, and 250 stimuli are delivered to the PMC. A figure of eight stimulation coil is used, and the rTMS unit used is a Magstim 200 stimulator (Magstim, Whitland, UK). The stimulation site for PMC stimulation is determined to be 2 cm anterior and 1 cm medial to the hotspot, estimated from the dorsal PMC established in a PET study as described in Fink et al. 1997 J. of Neurophysiol 77:2164-74. Stimuli are set to 80% of the resting motor threshold. To determine the motor hotspot, the stimulation coil is placed over the area 2 cm anterior to Cz (international 10-20 system) with the handle pointing backward and parallel to the midline. The intensity of stimulation is increased from 30% of the maximum output of the stimulator in 5% steps until an MEP just became visible. The coil is then moved in 0.5 cm steps in four directions (medial, lateral, posterior and anterior) until the maximum MEP was found. The coil is then removed and the position is marked on the subject's scalp as the motor hotspot. The resting motor threshold is defined by decreasing or increasing the stimulus intensity in 1% steps, as the minimum intensity that produces MEPs greater than 50 V.

Patients are treated with a treatment course of a first portion of three treatment regimens per week (Monday-Wednesday-Friday) for a period of twelve weeks, and a second portion including two treatment regimens per week (Tuesday-Thursday) for twelve weeks.

Evaluation of the torticollis (cervical dystonia) is assessed clinically via assessment of reduction of spasms causing forward (anterocollis), backwards (retrocollis), and sideways (torticollis) movements of the neck, and reduced jerking on movement. Patients' pain and discomfort related to their dystonia are also evaluated. Patients are also asked to report the subjective rating of their symptoms after rTMS into five grades: improvement, slight improvement, no change, slight deterioration, and deterioration. Patients are evaluated at the end of each treatment regimen, one hour after the end of each treatment regimen, and ten hours at the end of each treatment regimen. Results are statistically analyzed to determine whether findings are significant. Improvement or slight improvement is reported by the majority of the patients and in 11% of the sham patients. Results showed that as the treatment course progresses, the number of patients showing clinical improvement increases. Additionally, responses to rTMS are found to last for longer periods of time post each treatment regimen.

### Example 4

### Essential Tremor

Several patients having a mean age of about forty years of age having symptoms of essential tremor including tremors occurring upon engagement in a voluntary movement, such as drinking a glass of water, writing or threading a needle are selected for the study. Tremors appear especially in use of fine-motor skills such as using utensils or small tools. Tremors are not linked to any underlying disease or dysfunction. The average duration of the condition is about eight years. Patients are taking no medication at the time of the study. Studies are carried out at least one week post injection. The inclusion criterion for the study is adult onset with no family history. Patients with drug induced tremor are excluded from the study.

All patients have rTMS at a stimulation site over the premotor cortex or cerebellum, with a sham coil over the premotor cortex or cerebellum. The subjects are seated comfortably in a reclining chair and told to relax. Motor evoked potentials (MEP) are recorded with silver chloride disc electrodes, 1 cm in diameter, placed over the muscle belly and tendon of the affected muscle(s). Electromyography (EMG) signals are amplified, analogue-filtered, by an amplifier, and acquired at a sampling rate of 5 kHz. Monophasic rTMS is applied at 0.2 Hz, and 250 stimuli are delivered to the PMC. A figure of eight stimulation coil is used, and the rTMS unit used is a Magstim 200 stimulator (Magstim, Whitland, UK). The stimulation site for cerebellar stimulation is 2 cm below the inion. The stimulation site for PMC stimulation is determined to be 2 cm anterior and 1 cm medial to the hotspot, estimated from the dorsal PMC established in a PET study as described in Fink et al. 1997 J. of Neurophysiol 77:2164-74. Stimuli are set to 80% of the resting motor threshold. To determine the motor hotspot, the stimulation coil is placed over the area 2 cm anterior to Cz (international 10-20 system) with the handle pointing backward and parallel to the midline. The intensity of stimulation is increased from 30% of the maximum output of the stimulator in 5% steps until an MEP just became visible. The coil is then moved in 0.5 cm steps in four directions (medial, lateral, posterior and anterior) until the maximum MEP was found. The coil is then removed and the position is marked on the subject's scalp as the motor hotspot. The resting motor threshold is defined by decreasing or increasing the stimulus intensity in 1% steps, as the minimum intensity that produces MEPs greater than 50 V.

Patients are treated with a treatment course of a first portion of three treatment regimens per week (Monday-Wednesday-Friday) for a period of twelve weeks, and a second portion including two treatment regimens per week (Tuesday-Thursday) for twelve weeks.

Evaluation of the tremor is assessed clinically via assessment of reduction of tremors occurring upon engagement in a voluntary movement, such as drinking a glass of water, writing or threading a needle. Patients are also asked to report the subjective rating of their symptoms after rTMS into five grades: improvement, slight improvement, no change, slight deterioration, and deterioration. Patients are evaluated at the end of each treatment regimen, one hour after the end of each treatment regimen, and ten hours at the end of each treatment regimen. Results are statistically analyzed to determine whether findings are significant. Improvement or slight improvement is reported by the majority of the patients and in 11% of the sham patients. Results showed that as the treatment course progresses, the number of patients showing clinical improvement increases. Additionally, responses to rTMS are found to last for longer periods of time post each treatment regimen.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

What is claimed is:

1. A method for using transcranial magnetic stimulation (rTMS) to treat a particular muscle grouping involved in a movement disorder comprising treating a patient with a treatment course comprising a plurality of treatment regimens, each regimen comprising applying rTMS over a position on the patient's scalp corresponding to a cortical region controlling the muscle grouping.

2. The method of claim 1, wherein the movement disorder is selected from the group consisting of focal dystonia, generalized dystonia, a post-stroke movement disorder, a parkinsonian tremor, and essential tremor.

3. The method of claim 2, wherein the movement disorder is generalized or focal dystonia and the focus is the hand.

4. The method of claim 3, wherein the movement disorder is writer's cramp.

5. The method of claim 3, wherein the movement disorder is YIPS.

6. The method of claim 2, wherein the movement disorder is generalized or focal dystonia and the focus is the neck.

7. The method of claim 6, wherein the movement disorder is torticollis.

8. The method of claim 2, wherein the movement disorder is generalized or focal dystonia and the focus is the vocal cords.

9. The method of claim 8, wherein the movement disorder is spasmodic dysphonia.

10. The method of claim 1, wherein the treatment course comprises at least two treatment regimens within a 7 day period.

11. The method of claim 1, wherein the treatment course comprises at least three treatment regimens within a 7 day period.

12. The method of claim 1, wherein the treatment course comprises at least four treatment regimens within a 7 day period.

13. The method of claim 1, wherein the treatment course comprises at least five treatment regimens within a 7 day period.

14. The method of claim 10, wherein the treatment course further comprises at least two 7 day periods.

15. The method of claim 10, wherein the treatment course further comprises at least four 7 day periods.

16. The method of claim 10, wherein the treatment course further comprises at least eight 7 day periods.

17. The method of claim 10, wherein the treatment course further comprises at least twelve 7 day periods.

18. The method of claim 10, wherein the treatment course comprises at least three treatment regimens within a 7 day period for between four 7 day periods and 12 seven day periods, followed by at least two treatment regimens within a 7 day period for between four 7 day periods and 12 seven day periods.

19. The method of claim 10, wherein the treatment course comprises at least five treatment regimens within a 7 day period for between four 7 day periods and 12 seven day periods, followed by at least three treatment regimens within a 7 day period for between four 7 day periods and 12 seven day periods.

20. The method of claim 1, wherein the frequency of the rTMS stimulation during the treatment regimen is adjusted to provide an inhibitory effect to motor activation by the cortex.

21. The method of claim 20, wherein the rTMS has a frequency of between about 0.2 Hz and about 1 Hz.

22. The method of claim 20, wherein the rTMS has a frequency of between about 0.2 Hz and about 0.5 Hz.

23. The method of claim 1, wherein the rTMS treatment regimen has a duration between about 10 minutes and about 120 minutes.

24. The method of claim 1, wherein the rTMS treatment regimen has a duration between about 20 minutes and about 90 minutes.

25. The method of claim 1, wherein the rTMS treatment regimen has a duration between about 30 minutes and about 60 minutes.

26. The method of claim 1, wherein the cortical region is the premotor cortex.

27. The method of claim 1, wherein the cortical region is the supplementary motor area.

28. The method of claim 1, wherein the position of the coil of the rTMS device in a treatment regimen is determined by a method comprising: determining the motor hot spot for a particular muscle grouping comprising determining the position over the scalp where magnetic stimulation provoked the largest motor evoked potential (MEP) of the particular muscle grouping; and positioning the coil approximately 2.5 cm anterior to motor hot spot.

29. The method of claim 28, wherein the magnetic field strength used for a treatment regimen is 90% of the resting motor threshold (the minimum stimulation intensity over the motor hot spot that could elicit an MEP of no less than 50 microV in 5 of 10 trials.)

30. The method of claim 1, wherein the patient is a human.

31. The method of claim 1, wherein the treatment course leads to an improvement in a symptom of the movement disorder.

32. The method of claim 31, wherein the improvement in a symptom of the movement disorder lasts for longer than one hour.

33. The method of claim 31, wherein the improvement in a symptom of the movement disorder lasts for longer than four hours.

34. The method of claim 31, wherein the improvement in a symptom of the movement disorder lasts for longer than ten hours.

35. The method of claim 1, wherein the movement disorder is progressive and the treatment course leads to a slowing in the time course of the progression of the movement disorder.
